(19) United States
(12) Patent Application Publication
(10) Pub. No.: US 2022/0110953 A1
(43) Pub. Date: Apr. 14, 2022

(54) METHODS AND COMPOSITIONS FOR TREATING HUMAN PAPILLOMAVIRUS (HPV)-INDUCED CANCERS

(71) Applicant: The Board of Regents of the University of Oklahoma, Norman, OK (US)

(72) Inventor: Mojgan Padash Barmchi, Norman, OK (US)

(21) Appl. No.: 17/501,667

(22) Filed: Oct. 14, 2021

Related U.S. Application Data
(60) Provisional application No. 63/091,720, filed on Oct. 14, 2020.

(51) Int. Cl. A61K 31/665; A61P 35/00

(52) U.S. Cl. CPC A61K 31/665; A61P 35/00

(57) ABSTRACT

A method of treating a human papillomavirus (HPV)-induced cancer in a subject in need of such treatment by administering to the subject an effective amount of an inhibitor of Inhibitor of Nuclear Factor kappa-B kinase subunit beta (IKKβ). A method of inhibiting growth of human papillomavirus (HPV)-induced cancer cells, in vivo or in vitro, comprising exposing the cells to an inhibitor of Inhibitor of Nuclear Factor kappa-B kinase subunit beta (IKKβ).

### CROSS REFERENCE TO RELATED APPLICATIONS AND INCORPORATION BY REFERENCE STATEMENT

The present patent application claims priority under 37 CFR § 119(e) to U.S. Provisional Patent Application Ser. No. 63/091,720, filed on Oct. 14, 2020, the entire contents of which are hereby expressly incorporated herein by reference.

### BACKGROUND

Human papillomaviruses (HPV) are small, double-stranded circular DNA viruses that are categorized into two classes: low-risk HPVs and high-risk HPVs. The former directs skin warts and are not cancerous. The latter are carcinogenic, causing most cervical cancers as well as strongly implicated in many head-neck, oropharyngeal, anal, vulvar, vaginal and penile cancers. Among all high-risk HPVs, HPV 16 and HPV 18 are the most prominent types, causing more than 70% of all invasive cervical cancers. According to World Health Organization (WHO), every year 570,000 women are diagnosed with cervical cancer with a current mortality rate of more than 50%. The availability of effective vaccines against the most prevalent high-risk HPVs is expected to eventually reduce HPV-dependent tumors. However, the number of new HPV-induced cancer cases is not predicted to appreciably decline for the next few decades. Economic and cultural barriers hinder widespread immunization in middle and low-income, countries that account for the majority of cervical cancers. Further, chronic HPV infection can require several decades to provoke transformation.

HPV 16 and HPV 18 direct cellular transformation through the persistent expression of two viral early genes, E6 and E7. E6 and E7 oncoproteins cause cellular transformation through elimination of key tumor suppressors Rb and P53, respectively. High-risk HPV E6s contain a PDZ binding motif (PBM) at the extreme C terminus that is absent in low risk E6s. Interaction of the PBM with the PDZ domains of key host cellular PDZ domain proteins, including Magi, Dlg and Scribble, targets these proteins for ubiquitination and subsequent proteasome-mediated destruction. This action of E6 requires the assistance of the host E3 ubiquitin ligase, UBE3A and is necessary for cellular transformation. Transgenic mice deficient in the E6 PBM lack the ability to induce cellular transformation. The failure to induce cellular transformation is independent of P53, as PBM-deleted E6 retains the ability to inactivate P53. However, beyond the cellular targets of HPV oncogenes, we have a limited understanding of how persistent expression of E6 and E7 can lead to dysplasia and cancer.

Existing vaccines are only prophylactic against new HPV infections and are not effective against preexisting HPV infections, nor can they inhibit cancer progression and malignancy. Current treatments for invasive HPV-induced cancers are primarily radiation and chemotherapy, which show limited effectiveness. Furthermore survival rates of patients with advanced-stage cervical cancer are low. As a result of these hurdles, effective treatments of HPV-induced cancers in general, including but not limited to HPV-induced cervical head and neck, mouth, tongue, oropharyngeal, anal, vulvar, vaginal and penile cancers, remain a major unmet clinical need. It is to this unmet need that the present disclosure is directed.

### BRIEF DESCRIPTION OF THE DRAWINGS

FIG. 1 shows that reduction of IKKβ suppresses the E6 and UBE3A co-expression phenotypes (A) adult eye expressing GMR-Gal4 showing an intact eye morphology. (B) GMR-Gal4-driven co-expression of E6 and UBE3A causes rough eye morphology. (C) One mutated copy of IKKβ gene in GMR-Gal4 expressing eye has no effect on the eye morphology. (D) One mutated copy of IKKβ gene suppresses the E6+UBE3A-induced rough eye defects. (E) Expression of IKKβ RNAi suppresses the E6+UBE3A-induced rough eye defects.

FIG. 2 shows that inhibition of Human IKKβ in cervical cancer cells blocks their growth. Five different concentrations (250, 500, 750, 1000, 1250 nM) of human IKKβ inhibitor, IMD 0354, were applied to HPV-negative (HaCat), HPV 18-positive (HeLa), and HPV 16-positive (CaSki and SiHa) cells and the effect was measured by number of surviving cells after 48h. The inhibitor blocks the growth of HPV 16 and 18 cervical cancer cells, showing a most significant effect on HeLa cells: reducing the initial number of cells to 40%, at starting concentration of 250 nM and 10% at 1250 nM.

FIG. 3 shows that reduction of IKKβ suppresses the cellular abnormalities and restores the cell polarity and junctional integrity disrupted by E6+UBE3A co-expression. (A-C) Pupal eyes showing E-Cad immunolabeling. (A) Expression of GMR-Gal4, showing a normal stereotype pattern of ommatidia. (B) Co-expression of E6 and UBE3A causes severe abnormalities, including increase in number of pigment cells, bristle cells, and fusion of neighboring ommatidia. (C) A mutated copy of the IKKβ gene suppresses the E6+UBE3A abnormalities. (E-H′) Co-expression of E6 and UBE3A causes disruption of cell polarity and junctional complex, as shown by loss of Bazooka (Baz) and E-Cad from photoreceptor cells. This is in comparison with (D-G′) where Baz and E-Cad are both localized correctly in each photoreceptor. (F-I′) A mutated copy of IKKβ gene in eyes expressing E6 and UBE3A restores the polarity and junctional integrity as is shown by the correct localization of Baz and E-Cad. Scale bars indicate 10 μm. Insets are digitally magnified 200%.

FIG. 4 shows that reduction of IKKβ causes hyperphosphorylation of E6 and suppression of E6+UBE3A-mediated degradation of PDZ domain protein, Magi. (A-C) pupal eyes showing Magi immunolabeling. (B) Co-expression of E6+UBE3A results in loss of Magi from cone cells and pigment cells compared with (A) in which only the Gal4 driver is expressed. (C) A mutated copy of the IKKβ gene suppresses the E6+UBE3A-mediated loss of Magi. (D) Western blot of HeLa cell extracts shows that inhibition of IKKβ using a concentration of 500 nM of the inhibitor IMD 0354 results in phosphorylation of E6; compare that to lack of E6 phosphorylation in HeLa cells grown with no inhibitor (untreated) or with a low concentration of 100 nM. Scale bar indicates 10 μm.

FIG. 5 shows that reduction of IKKβ suppresses the cellular transformation caused by cooperative action of E6+UBE3A. (A, B) Expression of transgenes at 25° C. (B) A mutated copy of IKKβ gene in eyes expressing E6, UBE3A and an oncogenic Ras, suppresses pupal lethality and the severe defects caused by cooperative action of E6, UBE3A and oncogenic Ras (A). (C-E) Expression of transgenes at 22° C. (C) expression of oncogenic Ras in the eye causes cellular transformation. (D) Co-expression of E6 and UBE3A with oncogenic Ras enhances the Ras phenotype. (E) A mutated copy of IKKβ gene suppresses the severe eye abnormalities mediated by the synergistic effect of oncogenic Ras, E6 and UBE3A.

FIG. 6 shows that auranofin blocks cell growth in several human cervical cancer cell lines.

FIG. 7 shows the effect of Auranofin on tumor volume of cervical cancer xenografts. Mice harboring SiHa cervical cancer xenografts (HPV 16⁺) were treated with 10 mg/kg/day Auranofin or placebo. Tumor volume was monitored over time and compared between the two treatment groups.

FIG. 8 shows the effect of Auranofin on body weight in mice with cervical cancer xenografts. Mice harboring SiHa cervical cancer xenografts (HPV 16⁺) were treated with 10 mg/kg/day Auranofin or placebo. Body weigh was monitored over time and compared between the two treatment groups.

FIG. 9 shows the effect of Auranofin on final tumor weight of cervical cancer xenografts. Mice harboring SiHa cervical cancer xenografts (HPV 16⁺) were treated with 10 mg/kg/day Auranofin or placebo. Final tumor weight was measured on the last day of treatment and compared between the two treatment groups.

### DETAILED DESCRIPTION

In at least one embodiment, the present disclosure is directed to a method of treating a human papillomavirus (HPV)-induced cancer in a subject by administering to the subject an effective amount of an inhibitor of Inhibitor of Nuclear Factor kappa-B kinase subunit beta (IKK beta, IKKβ). Examples of such inhibitors include, but are not limited to, ACHP (IKK inhibitor VIII), Ainsliadimer A, anti-IKKβ antibody clone 10AG2 (and fragments and chimeric versions thereof), auranofin, Bay 65-1942, BI605906 (BIX02514), BMS-345541, BOT-64, CDDO-Me, IKK16 (IKK inhibitor VII), IMD-0354, IMD-1041, LY2409881, MLN120B, PF-184, PHA-408, PS-1145, SC-514, TPCA-1, Wedelactone, and Withaferin A (e.g., see Table 1 of Prescott, J. A., and S. J. Cook “Targeting IKKβ in Cancer: Challenges and Opportunities for the Therapeutic Utilisation of IKKβ Inhibitors,” Cells 2018, 7:115; doi 10.3390). Other examples of IKKβ inhibitors are described hereinbelow. Examples of HPV-induced cancers include but are not limited to cervical, head and neck, mouth, tongue, oropharyngeal, anal, vulvar, vaginal and penile cancers.

Drosophila has proven a strongly useful platform for modeling human diseases including cancer, owing in part to high conservation of genes and signaling pathways and availability of a broad array of genetic tools. One particular advantage of Drosophila disease models is their use in functional genetic screens designed to discover novel targets and pathways that mediate human disease. The present work used a recently developed Drosophila model of HPV 18 E6+UBE3A (U.S. Patent Publication 2018/0044743, the entirety of which is incorporated by reference herein) in a screen to identify kinases active in aspects of E6/UBE3A-induced transformation. The present work shows that reduced activity of Inhibitor of Nuclear Factor kappa-B kinase subunit beta (IKKβ) (a regulator of the innate immune response) strongly suppressed E6+UBE3A-mediated cellular abnormalities as well as rescuing degradation of E6 targets. Provide herein is evidence that the IKKβ-mediated suppression of Magi degradation is due to phosphorylation of E6, which was previously shown to block the interaction of E6 with PDZ domain proteins. Further, reduction in IKKβ suppressed the cellular transformation caused by the cooperative action of HPVE6 and oncogenic Ras. Finally, targeted inhibitors of IKKβ demonstrate that reduced IKKβ activity results in strongly reduced growth of cultured human cervical cancer cells and tumor volume of mice tumor xenografts. Thus, inhibitors of IKKβ can be used as therapeutics for HPV-induced cervical cancer.

Before further describing various embodiments of the compositions, kits, and methods of the present disclosure in more detail by way of exemplary description, examples, and results, it is to be understood that the present disclosure is not limited in application to the details of methods and compositions as set forth in the following description. The description provided herein is intended for purposes of illustration only and is not intended to be construed in a limiting sense. The inventive concepts of the present disclosure are capable of other embodiments or of being practiced or carried out in various ways. As such, the language used herein is intended to be given the broadest possible scope and meaning; and the embodiments are meant to be exemplary, not exhaustive. Also, it is to be understood that the phraseology and terminology employed herein is for the purpose of description and should not be regarded as limiting unless otherwise indicated as so. Moreover, in the following detailed description, numerous specific details are set forth in order to provide a more thorough understanding of the disclosure. However, it will be apparent to a person having ordinary skill in the art that the present disclosure may be practiced without these specific details. In other instances, features which are well known to persons of ordinary skill in the art have not been described in detail to avoid unnecessary complication of the description. It is intended that all alternatives, substitutions, modifications, and equivalents apparent to those having ordinary skill in the art are included within the scope of the present disclosure as defined herein. Thus, while the compositions and methods of the present disclosure have been described in terms of particular embodiments, it will be apparent to those of skill in the art that variations may be applied to the compositions and/or methods and in the steps or in the sequence of steps of the method described herein without departing from the concept, spirit, and scope of the inventive concepts.

All patents, published patent applications, and non-patent publications mentioned in the specification are indicative of the level of skill of those skilled in the art to which the present disclosure pertains. All patents, published patent applications, and non-patent publications referenced in any portion of this application are herein expressly incorporated by reference in their entirety to the same extent as if each individual patent or publication was specifically and individually indicated to be incorporated by reference.

Unless otherwise defined herein, scientific and technical terms used in connection with the present disclosure shall have the meanings that are commonly understood by those having ordinary skill in the art. Further, unless otherwise required by context, singular terms shall include pluralities and plural terms shall include the singular. Where used herein, the specific term “single” is limited to only “one.”

As utilized in accordance with the methods, compounds, and compositions of the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. Use of the word “we” as a pronoun herein refers generally to laboratory personnel or other contributors who assisted in laboratory procedures and data collection and is not intended to represent an inventorship role by said laboratory personnel or other contributors in any subject matter disclosed herein.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Throughout this application, the terms “about” or “approximately” are used to indicate that a value includes the inherent variation of error for the composition, the method used to administer the composition, or the variation that exists among the study subjects. As used herein the qualifiers “about” or “approximately” are intended to include not only the exact value, amount, degree, orientation, or other qualified characteristic or value, but are intended to include some slight variations due to measuring error, manufacturing tolerances, stress exerted on various parts or components, observer error, wear and tear, and combinations thereof, for example. The term “about” or “approximately,” where used herein when referring to a measurable value such as an amount, a temporal duration, and the like, is meant to encompass, for example, variations of ±20% or ±10%, or ±5%, or ±1%, or ±0.1% from the specified value, as such variations are appropriate to perform the disclosed methods and as understood by persons having ordinary skill in the art. As used herein, the term “substantially” means that the subsequently described event or circumstance completely occurs or that the subsequently described event or circumstance occurs to a great extent or degree. For example, the term “substantially” means that the subsequently described event or circumstance occurs at least 90% of the time, or at least 95% of the time, or at least 98% of the time.

As used herein any reference to “one embodiment” or “an embodiment” means that a particular element, component, step, feature, structure, or characteristic described in connection with the embodiment is included in at least one embodiment. The appearances of the phrase “in one embodiment” in various places in the specification are not necessarily all referring to the same embodiment.

When two or more active agents described in present disclosure, or their equivalents, are administered, they may be used or administered conjointly. As used herein the terms “conjointly” or “conjoint administration” refers to any form of administration of two or more different biologically-active compounds (i.e., active agents) such that the second compound is administered while the previously administered therapeutic compound is still effective in the body, whereby the two or more compounds are simultaneously active in the patient. For example, the different therapeutic compounds can be administered either in the same formulation, or in separate formulations, either concomitantly (together) or sequentially. When administered sequentially the different compounds may be administered immediately in succession, or separated by a suitable duration of time, as long as the active agents function together in a synergistic manner. In certain embodiments, the different therapeutic compounds can be administered within one hour of each other, within two hours of each other, within 3 hours of each other, within 6 hours of each other, within 12 hours of each other, within 24 hours of each other, within 36 hours of each other, within 48 hours of each other, within 72 hours of each other, or more. Thus an individual who receives such treatment can benefit from a combined effect of the different therapeutic compounds.

The term “pharmaceutically acceptable” refers to compounds and compositions which are suitable for administration to humans and/or animals without undue adverse side effects such as toxicity, irritation and/or allergic response commensurate with a reasonable benefit/risk ratio.

By “biologically active” is meant the ability of an agent to modify the physiological system of an organism without reference to how the agent (“active agent”) has its physiological effects.

As used herein, “pure” or “substantially pure” means an object species is the predominant species present (i.e., on a molar basis it is more abundant than any other object species in the composition thereof), and particularly a substantially purified fraction is a composition wherein the object species comprises at least about 50 percent (on a molar basis) of all macromolecular species present. Generally, a substantially pure composition will comprise more than about 80% of all macromolecular species present in the composition, more particularly more than about 85%, more than about 90%, more than about 95%, or more than about 99%. The term “pure” or “substantially pure” also refers to preparations where the object species (e.g., the peptide compound) is at least 60% (w/w) pure, or at least 70% (w/w) pure, or at least 75% (w/w) pure, or at least 80% (w/w) pure, or at least 85% (w/w) pure, or at least 90% (w/w) pure, or at least 92% (w/w) pure, or at least 95% (w/w) pure, or at least 96% (w/w) pure, or at least 97% (w/w) pure, or at least 98% (w/w) pure, or at least 99% (w/w) pure, or 100% (w/w) pure.

The terms “subject” and “patient” are used interchangeably herein and will be understood to refer to a warm-blooded animal, particularly a mammal, and more particularly, humans. Animals which fall within the scope of the term “subject” as used herein include, but are not limited to, dogs, cats, rats, mice, guinea pigs, chinchillas, horses, goats, ruminants such as cattle, sheep, swine, poultry such as chickens, geese, ducks, and turkeys, zoo animals, Old and New World monkeys, and non-human primates.

“Treatment” refers to therapeutic treatments, such as for promoting wound healing. “Prevention” refers to prophylactic or preventative treatment measures. The term “treating” refers to administering the composition to a patient for therapeutic purposes such as for promoting wound healing.

The terms “therapeutic composition” and “pharmaceutical composition” refer to an active agent-containing composition that may be administered to a subject by any method known in the art or otherwise contemplated herein, wherein administration of the composition brings about a therapeutic effect as described elsewhere herein. In addition, the compositions of the present disclosure may be designed to provide delayed, controlled, extended, and/or sustained release using formulation techniques which are well known in the art.

The term “effective amount” refers to an amount of an active agent (e.g., an IKK-beta inhibitor) which is sufficient to exhibit a detectable therapeutic effect without excessive adverse side effects (such as toxicity, irritation, and allergic response) commensurate with a reasonable benefit/risk ratio when used in the manner of the inventive concepts. The therapeutic effect may include, for example but not by way of limitation, a partial or complete elimination of an HPV-induced cancer. The effective amount for a patient will depend upon the type of patient, the patient's size and health, the nature and severity of the condition to be treated, the method of administration, the duration of treatment, the nature of concurrent therapy (if any), the specific formulations employed, and the like. The effective amount for a given situation can be determined by one of ordinary skill in the art based on the information provided herein.

The term “ameliorate” means a detectable or measurable improvement in a subject's condition, disease, or symptom thereof. A detectable or measurable improvement includes a subjective or objective decrease, reduction, inhibition, closure, suppression, limit, or control in the occurrence, frequency, severity, progression, or duration of the condition or disease, or an improvement in a symptom or an underlying cause or a consequence of the disease, or a reversal of the disease. A successful treatment outcome can lead to a “therapeutic effect” or “benefit” of completely or partially decreasing, reducing, inhibiting, suppressing, limiting, controlling, or preventing the occurrence, frequency, severity, progression, or duration of a disease or condition, or consequences of the disease or condition.

A decrease or reduction in the worsening of a disease or condition, such as stabilizing the condition or disease, such as a tumor, is also a successful treatment outcome. A therapeutic benefit therefore need not be complete ablation or reversal of the disease or condition, or of any one of, or most, or all adverse symptoms, complications, consequences, or underlying causes associated with the disease or condition. Thus, a satisfactory endpoint may be achieved when there is an incremental improvement such as a partial decrease, reduction, inhibition, suppression, limit, control, or prevention in the occurrence, frequency, severity, progression, or duration, or inhibition or reversal of the condition or disease (e.g., stabilizing), over a short or long duration of time (hours, days, weeks, months, etc.), such as partial closure of a wound. Effectiveness of a method or use, such as a treatment that provides a potential therapeutic benefit or improvement of a condition or disease, can be ascertained by various methods, measurements, and testing assays.

In certain non-limiting embodiments, the dosage of the IKKβ inhibitor administered to a subject could be in a range of about 1 μg per kg of subject body mass to about 1000 mg/kg, or in a range of about 5 μg/kg to about 500 mg/kg, or in a range of about 10 μg/kg to about 300 mg/kg, or in a range of about 25 μg/kg to about 250 mg/kg, or in a range of about 50 μg/kg to about 250 mg/kg, or in a range of about 75 μg/kg to about 250 mg/kg, or in a range of about 100 μg/kg to about 250 mg/kg, or in a range of about 200 μg/kg to about 250 mg/kg, or in a range of about 300 μg/kg to about 250 mg/kg, or in a range of about 400 μg/kg to about 250 mg/kg, or in a range of about 500 μg/kg to about 250 mg/kg, or in a range of about 600 μg/kg to about 250 mg/kg, or in a range of about 700 μg/kg to about 250 mg/kg, or in a range of about 800 μg/kg to about 250 mg/kg, or in a range of about 900 μg/kg to about 250 mg/kg, or in a range of about 1 mg/kg to about 200 mg/kg, or in a range of about 1 mg/kg to about 150 mg/kg, or in a range of about 2 mg/kg to about 100 mg/kg, or in a range of about 5 mg/kg to about 100 mg/kg, or in a range of about 10 mg/kg to about 100 mg/kg, or in a range of about 25 mg per kg to about 75 mg/kg.

The one or more IKKβ inhibitors (alone, or used conjointly with another therapeutic) can be administered, for example but not by way of limitation, on a one-time basis, or administered at multiple times (for example but not by way of limitation, from one to five times per day, or once or twice per week), or continuously via a venous drip, depending on the desired therapeutic effect. In one non-limiting example of a therapeutic method of the present disclosure, the composition is provided in an IV infusion. Administration of the compounds used in the pharmaceutical composition or to practice the method of the present disclosure can be carried out in a variety of conventional ways, such as, but not limited to, topically, orally, by inhalation (e.g., intrabronchial, intranasal or oral inhalation, intranasal drops), rectally, or by cutaneous, subcutaneous, intraperitoneal, or parenteral (e.g., intravenous, intraarterial, intramuscular, subcutaneous injection) injection. Oral formulations may be formulated such that the compounds pass through a portion of the digestive system before being released, for example it may not be released until reaching the small intestine, or the colon. When the active agent is delivered by inhalation, it may be delivered via a soft mist nebulizer (e.g., a jet, ultrasonic, or vibrating-mesh nebulizer), a pressurized metered-dose inhaler (MDI), or a dry powder inhaler (DPI), or by any other suitable means, e.g., via a catheter inserted directly into the lung, or via a ventilator when a patient himself or herself is unable to inhale voluntarily.

In certain embodiments, the methods of the present disclosure include topical, transdermal, sub-dermal, enteral, parental or intravenous administration of an active agent (one or more IKKβ inhibitors). For example (but not by way of limitation), topical administration of the active agent may comprise the administration of a cream, gel, ointment, spray, lip-balm, balm, emulsion, liposome, liquid crystal preparation or lotion, or any combination thereof. In one embodiment, administration comprises an at least once a day administration for one or more days (e.g., about 1 to about 30 days) until at least one symptom of the inflammatory disease or condition is alleviated. In another embodiment, administration comprises an at least twice a day administration for one or more days (e.g., about 1 to about 30 days) until at least one symptom of the condition is alleviated. In another embodiment, administration comprises an at least about 3 to about 6 times per day administration for one or more days (e.g., about 1 to about 30 days) until at least one symptom of the condition is alleviated.

The composition for topical or internal application may be provided in any suitable solid, semi-solid, or liquid form. In certain embodiments, the topical composition may be provided in or be disposed in a carrier(s) or vehicle(s) such as, for example (but not by way of limitation), creams, pastes, gums, lotions, gels, foams, ointments, emulsions, suspensions, aqueous solutions, powders, lyophilized powders, solutions, granules, foams, drops, eye drops, adhesives, sutures, aerosols, sprays, sticks, soaps, bars of soap, balms, body washes, rinses, tinctures, gel beads, gauzes, wound dressings, bandages, cloths, towelettes, stents, and sponges. Non-limiting examples of formulations of such carriers and vehicles include, but are not limited to, those shown in “Remington, The Science and Practice of Pharmacy,” 22nd ed., 2012, edited by Loyd V. Allen, Jr.

Creams are emulsions of water in oil (w/o), or oil in water (o/w). O/w creams spread easily and do not leave the skin greasy and sticky. W/o creams tend to be more greasy and more emollient. Ointments are semi-solid preparations of hydrocarbons and the strong emollient effect makes it useful in cases of dry skin. The occlusive effect enhances penetration of the active agent and improves efficacy. Pastes are mixtures of powder and ointment. The addition of the powder improves porosity thus breathability. The addition of the powder to the ointment also increases consistency so the preparation is more difficult to rub off or contact non-affected areas of the skin. Lotions are liquid preparations in which inert or active medications are suspended or dissolved. For example, an o/w emulsion with a high water content gives the preparation a liquid consistency of a lotion. Most lotions are aqueous of hydroalcoholic systems wherein small amounts of alcohol are added to aid in solubilization of the active agent and to hasten evaporation of the solvent from the skin surface. Gels are transparent preparations containing cellulose ethers or carbomer in water, or a water-alcohol mixture. Gels liquefy on contact with the skin, dry, and leave a thin film of active medication.

In certain non-limiting embodiments, the composition may comprise the active agents in a concentration of, but is not limited to, about 0.0001 M to about 1 M, for example, or about 0.001 M to about 0.1 M. The composition may comprise about 0.01 to about 1000 milligrams of the active agents per ml of carrier or vehicle with which the active agents are combined in a composition or mixture. The composition may comprise about 1 wt % to about 90 wt % (or about 1 mass % to about 90 mass %) of one or more shikimate analogues and about 10 wt % to about 99 wt % (or about 10 mass % to about 99 mass %) of one or more secondary compounds (where “wt %” is defined as the percentage by weight of a particular compound in a solid or liquid composition, and “mass %” is defined as the percentage by mass of a particular compound in a solid or liquid composition).

The topical compositions may further comprise ingredients such as (but not limited to) propylene glycol, sodium stearate, glycerin, a surfactant (e.g., sodium laurate, sodium laureth sulfate, and/or sodium lauryl sulfate), and water, and optionally, sorbitol, sodium chloride, stearic acid, lauric acid, aloe vera leaf extract, pentasodium penetrate, and/or tetrasodium etidronate.

The topical compositions may be formulated with liquid or solid emollients, solvents, thickeners, or humectants. Emollients include, but are not limited to, stearyl alcohol, mink oil, cetyl alcohol, oleyl alcohol, isopropyl laurate, polyethylene glycol, olive oil, petroleum jelly, palmitic acid, oleic acid, and myristyl myristate. Emollients may also include natural butters extracted from various plants, trees, roots, or seeds. Examples of such butters include, but are not limited to, shea butter, cocoa butter, avocado butter, aloe butter, coffee butter, mango butter, or combination thereof.

Suitable materials which may be used in the compositions as carriers or vehicles or secondary compounds or solvents include, but are not limited to, propylene glycol, ethyl alcohol, isopropanol, acetone, diethylene glycol, ethylene glycol, dimethyl sulfoxide, and dimethyl formamide. Suitable humectants include, but are not limited to, acetyl arginine, algae extract, Aloe barbadensis leaf extract, 2,3-butanediol, chitosan lauroyl glycinate, diglycereth-7 malate, diglycerin, diglycol guanidine succinate, erythritol, fructose, glucose, glycerin, honey, hydrolyzed wheat protein/polyethylene glycol-20 acetate copolymer, hydroxypropyltrimonium hyaluronate, inositol, lactitol, maltitol, maltose, mannitol, mannose, methoxypolyethylene glycol, myristamidobutyl guanidine acetate, polyglyceryl sorbitol, potassium pyrollidone carboxylic acid (PCA), propylene glycol (PGA), sodium pyrollidone carboxylic acid (PCA), sorbitol, and sucrose. Other humectants may be used for yet additional embodiments of the compositions of the present disclosure.

Suitable thickeners include, but are not limited to, polysaccharides, in particular xantham gum, guar-guar, agar-agar, alginates, carboxymethylcellulose, relatively high molecular weight polyethylene glycol mono- and diesters of fatty acids, polyacrylates, polyvinyl alcohol and polyvinylpyrrolidone, surfactants such as, for example, ethoxylated fatty acid glycerides, esters of fatty acids with polyols such as, for example, pentaerythritol or trimethylpropane, fatty alcohol ethoxylates or alkyl oligoglucosides, and electrolytes, such as sodium chloride and ammonium chloride.

The topical compositions may further comprise one or more penetrants, compounds facilitating penetration of active ingredients into the skin of a patient. Non-limiting examples of suitable penetrants include isopropanol, polyoxyethylene ethers, terpenes, cis-fatty acids (oleic acid, palmitoleic acid), acetone, laurocapram dimethyl sulfoxide, 2-pyrrolidone, oleyl alcohol, glyceryl-3-stearate, cholesterol, myristic acid isopropyl ester, and propylene glycol. Additionally, the compositions may include surfactants or emulsifiers for forming emulsions. Either a water-in-oil or oil-in-water emulsion may be formulated. Examples of suitable emulsifiers include, but are not limited to, stearic acid, cetyl alcohol, PEG-100, stearate and glyceryl stearate, cetearyl glucoside, polysorbate 20, methylcellulose, sodium carboxymethylcellulose, glycerin, bentonite, ceteareth-20, cetyl alcohol, cetearyl alcohol, lanolin alcohol, riconyl alcohol, self-emulsifying wax (e.g., Lipowax P), cetyl palmitate, stearyl alcohol, lecithin, hydrogenated lecithin, steareth-2, steareth-20, and polyglyceryl-2 stearate.

When a therapeutically effective amount of the composition(s) is administered orally, it may be in the form of a solid or liquid preparation such as (but not by way of limitation) capsules, pills, tablets, lozenges, melts, powders, suspensions, solutions, elixirs or emulsions. Solid unit dosage forms can be capsules of the ordinary gelatin type containing, for example, surfactants, lubricants, and inert fillers such as lactose, sucrose, and cornstarch, or the dosage forms can be sustained release preparations. The pharmaceutical composition(s) may contain a solid carrier, such as a gelatin or an adjuvant. The tablet, capsule, and powder may contain from about 0.05 to about 95% of the active agent by dry weight. When administered in liquid form, a liquid carrier such as (but not limited to) water, petroleum, oils of animal or plant origin such as peanut oil, mineral oil, soybean oil, or sesame oil, or synthetic oils may be added. The liquid form of the pharmaceutical composition(s) may further contain physiological saline solution, dextrose or other saccharide solution, or glycols such as ethylene glycol, propylene glycol, or polyethylene glycol. When administered in liquid form, the pharmaceutical composition(s) particularly contains from about 0.005% to about 95% by weight of the active substance. For example (but not by way of limitation), a dose of about 10 mg to about 1000 mg once or twice a day could be administered orally.

In another non-limiting embodiment, the composition(s) of the present disclosure can be tableted with conventional tablet bases such as lactose, sucrose, and cornstarch in combination with binders, such as acacia, cornstarch, or gelatin, disintegrating agents such as potato starch or alginic acid, and a lubricant such as stearic acid or magnesium stearate. Liquid preparations are prepared by dissolving the composition(s) in an aqueous or non-aqueous pharmaceutically acceptable solvent which may also contain suspending agents, sweetening agents, flavoring agents, and preservative agents as are known in the art.

For parenteral administration, for example, the composition(s) may be dissolved in a physiologically acceptable pharmaceutical carrier and administered as either a solution or a suspension. Illustrative of suitable (but non-limiting) pharmaceutical carriers are water, saline, dextrose solutions, fructose solutions, ethanol, or oils of animal, vegetative, or synthetic origin. The pharmaceutical carrier may also contain preservatives and buffers as are known in the art.

When a therapeutically effective amount of the composition(s) is administered by intravenous, cutaneous, or subcutaneous injection, the active agent may be in the form of a pyrogen-free, parenterally acceptable aqueous solution or suspension. The preparation of such parenterally acceptable solutions, having due regard to pH, isotonicity, stability, and the like, is well within the skill in the art. A particular pharmaceutical composition for intravenous, cutaneous, or subcutaneous injection may contain, in addition to the active agent(s), an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical composition(s) of the present disclosure may also contain stabilizers, preservatives, buffers, antioxidants, or other additives known to those of skill in the art.

Additional pharmaceutical methods may be employed to control the duration of action of the composition(s). Increased half-life and/or controlled release preparations may be achieved through the use of polymers to conjugate, complex with, and/or absorb the active substances described herein. The controlled delivery and/or increased half-life may be achieved by selecting appropriate macromolecules (for example but not by way of limitation, polysaccharides, polyesters, polyamino acids, homopolymers polyvinyl pyrrolidone, ethylenevinylacetate, methylcellulose, or carboxymethylcellulose, and acrylamides such as N-(2-hydroxypropyl) methacrylamide), and the appropriate concentration of macromolecules as well as the methods of incorporation, in order to control release. The active agent(s) may also be ionically or covalently conjugated to the macromolecules described above, as long as they retain activity. Another possible method useful in controlling the duration of action of the composition(s) by controlled release preparations and half-life is incorporation of the composition(s) or functional derivatives thereof into particles of a polymeric material such as (but not limited to) polyesters, polyamides, polyamino acids, hydrogels, poly(lactic acid), ethylene vinylacetate copolymers, copolymer micelles of, for example, PEG and poly(l-aspartamide).

In at least one embodiment, the present disclosure is directed to a method of inhibiting growth of human papillomavirus (HPV)-induced cancer cells, in vivo or in vitro, by exposing the cells to an inhibitor of Inhibitor of Nuclear Factor kappa-B kinase subunit beta (IKKβ). In at least one embodiment, the present disclosure is directed to a method of treating a human papillomavirus (HPV)-induced cancer in a subject by administering to the subject an effective amount of an inhibitor of Inhibitor of Nuclear Factor kappa-B kinase subunit beta (IKK beta, IKKβ). The HPV-induced cancers which may be treated using the presently disclosed methods include, but are not limited to, cervical, head and neck, mouth, tongue, oropharyngeal, anal, vulvar, vaginal and penile cancers.

Examples of IKKβ inhibitors that can be used to inhibit growth of HPV-induced cancer cells and/or to treat HPV-induced cancers in patients include, but are not limited to, ACHP (IKK inhibitor VIII), Ainsliadimer A, anti-IKKβ antibody clone 10AG2 (or fragments or chimeric derivatives thereof), auranofin, Bay 651942, BI605906 (BIX02514), BMS-345541, BOT-64, CDDO-Me, IKK16 (IKK inhibitor VII). IMD-0354, IMD-1041, LY2409881, MLN120B, PF-184, PHA-408, PS-1145, SC-514, TPCA-1, Wedelactone, and Withaferin A. Other examples of IKKβ inhibitors that can be used herein include, but are not limited to, the IKKβ inhibitor compounds of examples 1-279 in U.S. Pat. No. 7,547,691; compounds of formulas 1A and 1B of US Patent Publications 20110046210, 20100087515, and 20100069473; dihydromyricetin of US Patent Publication 20120053235; Cyclopentyl(2S,4E)-2-amino-5-{3-[4-carbamoyl-5(carbamoylamino)-2-thienyl]-phenyl}pent-4-enoate, Cyclopentyl 5-{3-[4-carbamoyl-5-(carbamoylamino)-2-thienyl]phenyl}-L-norvalinate, Cyclopentyl(2S,4E)-2-amino-5-{3-[4-carbamoyl-5-(carbamoylamino)-2-thienyl-]-5-methylphenyl}pent-4-enoate, Cyclopentyl(25,4E)-2-amino-5-{5-[4-carbamoyl-5-(carbamoylamino)-2-thienyl-]-2-methylphenyl}pent-4-enoate, Cyclopentyl O-{3-[4-carbamoyl-5-(carbamoylamino)-2-thienyl]phenyl}-L-homoserinate, Cyclopentyl O-{3-[4-carbamoyl-5-(carbamoylamino)-2-thienyl]phenyl}-L-homoserinate, Cyclopentyl N-{3-[4-carbamoyl-5-(carbamoylamino)-2-thienyl]benzyl}-L-alaninate, and tert-Butyl N-{3-[4-carbamoyl-5-(carbamoylamino)-2-thienyl]benzyl}-L-alaninate of U.S. Patent Publication 20110039920; and compounds of U.S. Patent Publication 20040241166.

Certain novel embodiments of the present disclosure, having now been generally described, will be more readily understood by reference to the following examples, which are included merely for purposes of illustration of certain aspects and embodiments of the present disclosure, and are not intended to be limiting. The following detailed examples are to be construed, as noted above, only as illustrative, and not as limiting of the present disclosure in any way whatsoever. Those skilled in the art will promptly recognize appropriate variations from the various compositions, structures, components, procedures and methods.

### Example 1

### Material and Methods

### Fly Strains

The following fly stocks were used: UAS-hUBE3A, UAS-HPV18-E6, GMR-Gal4, Kinome set, IKKβ/TM6B, Tb, UAS-IKKβRNAi attp40, UAS-Ras64BV14 from Bloomington Drosophila stock center.

### In Vitro Assays

To examine the effect of IMD 0354 on the growth of HaCat (HPV⁻), HeLa (HPV 18⁺), CasKi and SiHa (both HPV 16⁺), the cells were plated out and allowed to adhere overnight. Next day their culture medium was replaced and different concentrations of IMD 0354 (final concentrations 250, 500, 750, 1000, 1250 nM, using DMSO to equalize input volumes and as negative control [0]) were added to the plates. Cells were counted 48h later.

### Biochemistry

HPV-18-positive HeLa cells were seeded on 6 cm dishes and allowed to attach overnight. Fresh medium was then added, containing either 0, 100 nM or 500 nM IMD0345 inhibitor (in DMSO). After 5 hours the cells were harvested in 2×SDS-PAGE gel loading buffer, run on SDS-PAGE, and analyzed by Western Blot. The membrane was probed with antibody specific for phosphorylated E6, as described previously (Boon and Banks 2012, DOI: 10.1128/JVI.02074-12). Following incubation with primary antibody, the appropriate horseradish peroxidase (HRP)-conjugated secondary antibodies (Dako) were used, followed by enhanced chemiluminescence (ECL) detection according to the manufacturer's instructions.

### Immunohistochemistry

For immunolabeling pupal eyes 40-42 hrs after puparium formation were dissected in PBS and fixed in 4% formaldehyde. Fixed tissues were washed three times in PBS solution containing 0.1% Triton-X-100 and blocked in 5% normal goat serum for 1 hour before incubation with primary antibodies. The primary antibodies used in this study were rabbit anti-Magi 1:200, rabbit anti-Baz 1:1000, rat anti-DE-cadherin 1:50 (Developmental Studies Hybridoma Bank). The appropriate secondary antibodies were con-jugated Alexa488, Alexa594, and Alexa 647 (Invitrogen).

### Results

### Kinome Screen Identified IKKβ as a Mediator of E6+hUBE3A-Mediated Defects

The fly eye is a compound eye consisting of 750 unit eyes. These ‘ommatidia’ are clusters of sensory neurons arranged in a precisely repeated hexagonal pattern due to precise arrangement of supporting pigment cells. Formation of this highly organized pattern requires precisely regulated cell proliferation, cell differentiation and programmed cell death; disruption of any of these processes leads to a disorganized, rough eye phenotype that is readily scored under a light microscope.

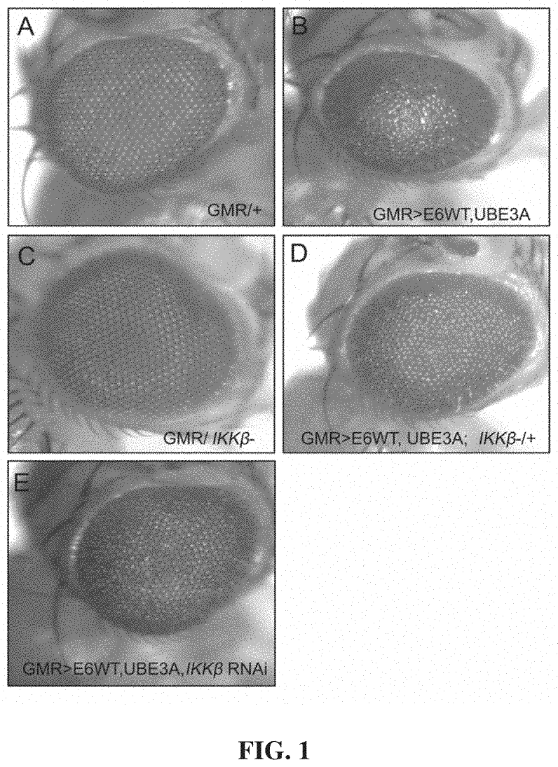

We have previously shown that co-expression of E6 and human UBE3A (hUBE3A) in the developing fly eye leads to a disorganized, rough eye phenotype. To identify loci that modify the E6+hUBE3A-induced eye defects, we used mutations in the kinome to perform a dominant genetic modifier screen. Flies with stable integration of the transgenes GAM-Gal4, UAS-E6, and UAS-hUBE3A (referred to as GMR>E6/hUBE3A) were crossed to flies heterozygous for a mutant kinase. Comparing GMR>E6/hUBE3A; kinase^{+/-} to GMR>E6/hUBE3A flies, we screened 195 kinases and identified IKKβ as the strongest suppressor of the E6+hUBE3A-mediated rough eye phenotype (FIG. 1D, compare with FIG. 1B; FIG. 1A, C are controls). Rescue was evident in 100% of flies (n=40) with GMR>E6/hUBE3A; IKKβ^{+/-} genotype and to the same extent shown in FIG. 1D. The rescue by reduced IKKβ activity was further confirmed with an RNA-interference transgene targeting IKKβ (FIG. 1E).

Inhibiting IKKβ in Cervical Cancer Cells Blocked their Growth

IKKβ is a serine/threonine kinase that is highly conserved between flies and humans. It regulates the innate immune pathway by activating NF-κB, which in turn activates the expression of antimicrobial peptides to fight against pathogens. To determine whether IKKβ also has a functional link to E6+UBE3A in human cells, we assessed the effect of the commercial IKKβ inhibitor IMD 0354 on the growth of HaCat (HPV⁻), HeLa (HPV 18⁺), CasKi (HPV 16⁺), and SiHa (HPV 16⁺) cells. Different concentrations of the inhibitor were tested ranging from 250, 500, 750, 1000, to 1250 nM.

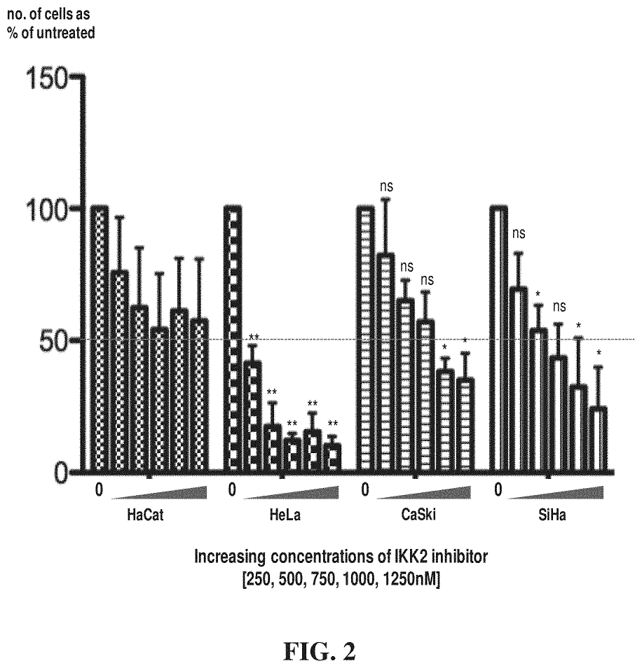

IMD 0354 reduced growth of all four cell types: HeLa cells were most strongly affected, with a significant effect starting at the lowest concentration of 250 nM. While the effect of the IMD 0354 was significant on the HPV 18 and 16 positive cells, it had a minor effect on the growth of the HaCat cells that lacked HPV (FIG. 2). These results suggested that the IKKβ-mediated mechanism of E6+UBE3A-induced cellular abnormalities is conserved between humans and fruit flies.

Reducing IKKβ Suppressed the Cellular Defects Caused by Co-Expression of E6 and hUBE3A

As reduction in IKKβ suppressed the rough eye phenotype caused by co-expression of E6 and hUBE3A, we examined the eye tissue of these flies at 40 hrs after puparium formation, the time point at which the E6+hUBE3A effect becomes apparent. In the normal pupal eye, each ommatidium consists of eight photoreceptor cells covered by four glial-like cells or cone cells and two primary pigment cells. Neighboring ommatidia are separated from each other by a lattice of secondary and tertiary pigment cells and the sensory bristle cells. This interweaving lattice of pigment cells organizes the ommatidial array into a precise pattern of repeated hexagons.

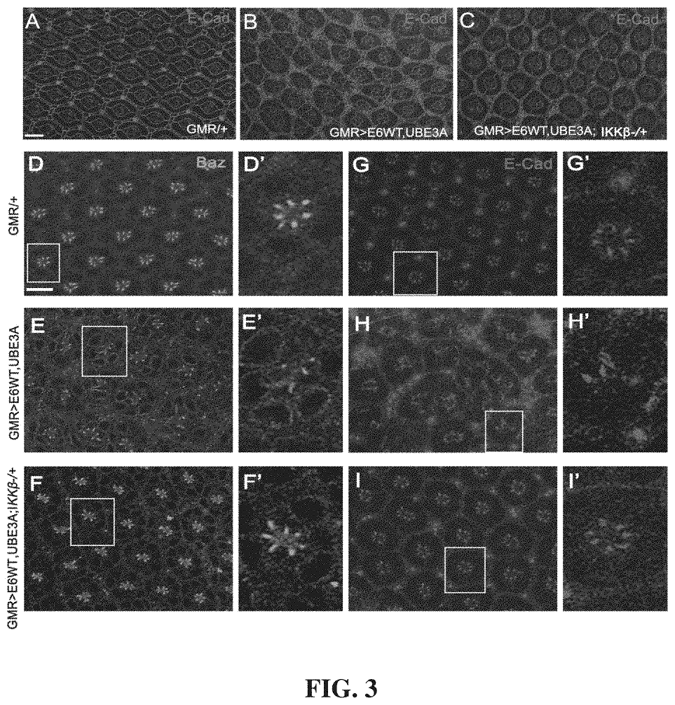

Pupal eyes expressing E6 and hUBE3A exhibit severe morphological defects, including fusion of neighboring ommatidia, increase in the number of pigment cells and cone cells and a severe alteration in the stereotyped pattern of ommatidia. In comparison, removing a single genomic copy of IKKβ (GMR>E6/hUBE3A; IKKβ^{+/-}) led to a strong phenotypic rescue: pigment and cone cell defects were reduced and the overall organization of the ommatidial array improved (FIG. 3 A-C). These observations suggest that E6 interferes with a molecular mechanism involving IKKβ and that this mechanism plays an important role in E6-induced cellular abnormalities.

Reducing IKKβ Suppressed the Junctional and Polarity Defects Caused by Co-Expression of E6 and hUBE3A

We have previously shown that E6, in cooperation with UBE3A, perturbs the integrity of junctional and polarity complexes. Therefore, we hypothesized that reducing IKKβ activity might suppress these defects. Immunolabeling for junctional marker E-cadherin and polarity marker Bazooka (the homolog of human Par-3) revealed that, in comparison with pupal eyes expressing E6 and UBE3A, in which both the junctional and polarity complexes were perturbed in ommatidia (FIG. 3 E-H′), GMR>E6/hUBE3A; IKKβ^{+/-} pupal eyes showed no disruption of junctional and polarity complexes and the integrity of these complexes was restored to the extend seen in control eye tissues (FIG. 3 F-I′ compared to D-G′). These observations suggest that alterations in IKKβ significantly contribute to the E6-induced cellular junctional and polarity disorganization.

### Reduced IKKβ Activity Suppressed E6+hUBEA-Induced Degradation of PDZ Domain Proteins

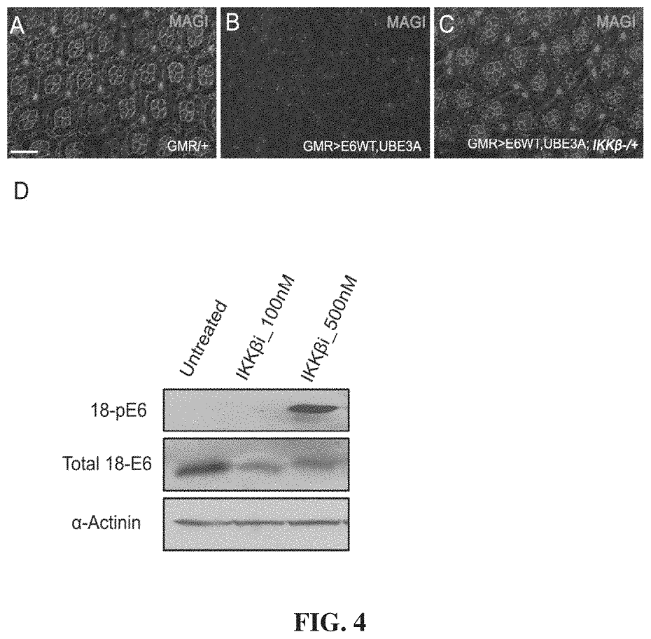

Proteasomal degradation of PDZ domain-containing proteins, including Magi, Dlg, and Scribble, was shown to be crucial for the cancerous effect of HPV 16 and 18 E6. We have previously shown that HPV 18 E6, with the addition of human UBE3A, targets the fly counterparts of these proteins for ubiquitin-mediated proteasomal destruction. As a reduction in IKKβ levels suppressed the cellular defects caused by E6 plus hUBE3A, we asked whether the E6-mediated degradation of PDZ domain proteins was altered. To address this question, we examined the level of Magi, as Magi has been identified as the major degradation target of E6 in human and Drosophila. Immunolabeling of pupal eyes for Magi revealed that, whereas GMR>E6/hUBE3A eyes exhibited a complete loss of Magi, GMR>E6/hUBE3A: IKKβ^{+/-} pupal eyes exhibited no detectable loss of Magi (FIG. 4A-C). This result suggests that reducing IKKβ activity suppresses the E6-induced degradation of Magi and that rescue of Magi degradation is likely to play a role in suppression of E6-induced cellular defects.

### Reducing IKKβ Resulted in Hyperphosphorylation of E6

Phosphorylation of the HPV 18 E6 PBM was previously demonstrated to block its interaction with PDZ domain proteins Dlg and Magi. To assess whether phosphorylation-mediated regulation of E6 plays a role in suppression of Magi degradation, we treated cells expressing HPV 18 E6 with the IKKβ inhibitor MID 0354 (at 100 and 500 nM) and compared with untreated cells for E6 phosphorylation. Western blot analysis was performed using an antibody to detect phosphorylated E6. We found that inhibition of IKKβ resulted in extensive phosphorylation of E6, which was absent in untreated cells (FIG. 4D). This result is consistent with previous findings, and suggests that the lack of Magi degradation in cells expressing E6+hUBE3A with mutated IKKβ could be due to the loss of PDZ recognition by E6.

### Reducing IKKβ Suppressed the Cooperative Effect of Ras and E6+hUBE3A

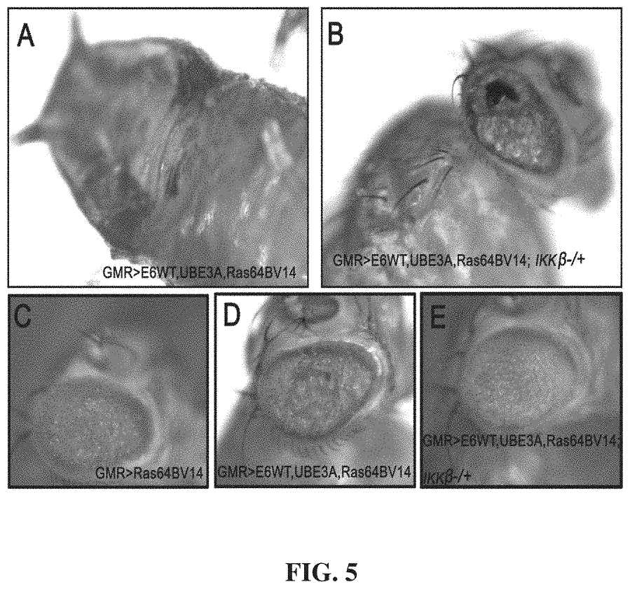

Previous studies have shown that the HPV oncogenes E6 and E7 alone are insufficient to direct oncogenic transformation, and that other factors including genetic alterations contribute to HPV-induced tumorigenesis in humans and mice. Mutations in Ras family proteins have been implicated in HPV-related cancers, and we have previously shown that cooperation between E6 and oncogenic isoforms of Drosophila Ras (Ras64B^{V14}) promotes cellular transformation and malignancy in fly epithelia. Therefore, we asked whether reducing IKKβ activity can suppress the cellular transformation caused by the cooperation of oncogenic Ras and E6. Co-expression of oncogenic Ras, Ras64B^{V14} with E6, and hUB3A in eye imaginal discs (GMR>E6/hUBE3A/Ras64B^{V14}) at 25° C. resulted in overgrowth and pupal lethality (FIG. 5A). However, when the level of IKKβ was reduced (GMR>E6/hUBE3A/Ras64B^{V14}; IKKβ^{+/-}), the lethality was countered and flies developed to adulthood. These adult flies, however, still exhibited significant abnormalities in eye morphology, suggesting incomplete rescue (FIG. 5B).

Gal4 activity is reduced at lower temperatures. At 22° C., GMR>E6/hUBE3A/Ras64B^{V14} flies developed to adulthood, exhibiting enhanced transformed eye morphology in comparison to expression of oncogenic Ras alone (FIG. 5D compared to FIG. 5C). Reducing IKKβ activity significantly suppressed the transformed eye morphology in these conditions (FIG. 5E), further demonstrating that IKKβ is important for the cooperative action of E6 and oncogenic Ras.

### Auranofin Inhibits Cervical Cancer Cells

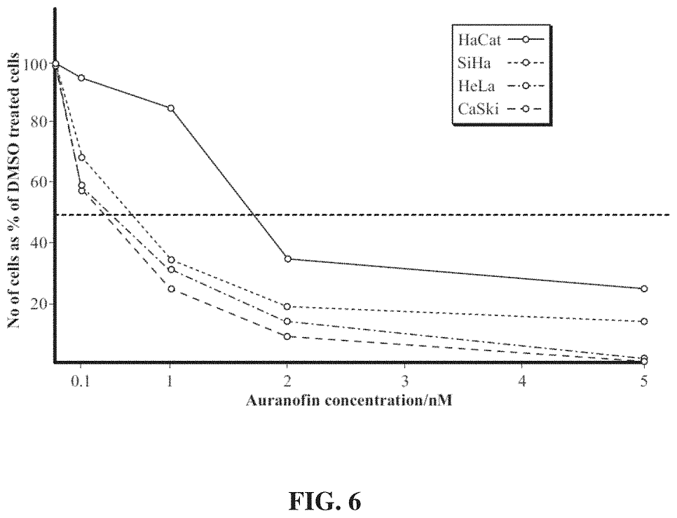

Auranofin is a compound which inhibits IKKβ by reacting with Cys-179 of IKKβ. Results shown in FIG. 6 demonstrate that Auranofin is active against HPV 16⁺ and HPV 18⁺ human cervical cancer cell lines. The translational potential for use of Auranofin for treatment of cervical cancer is enhanced by the extensive clinical data available for this compound through its use as an oral agent in treatment of arthritis and parasite infections. Currently, a major trend in anti-cancer drug development is to repurpose drugs that have been, or are currently being, used to treat other diseases. The availability of extensive data on clinical use of these agents speeds up and reduces the expense of gaining FDA approval for a new indication.

The results provided above identifies IKKβ as a mediator of the HPV 18 E6 and hUBE3A-induced cellular defects in both fly and human cancer models. HPV 16 E6 interacts with components of the innate immune pathway, including IKKβ, and activates the NF-κB transcription factor. IKKβ phosphorylates the inhibitor of NF-κB, resulting in its ubiquitination and proteasomal degradation. This action of IKKβ frees the NF-κB, which in turn enters the nucleus and activates the transcription of pro-inflammatory, pro-cell proliferation and anti-apoptotic genes. Increased expression of IKKβ and its association with an aggressive phenotype has been reported in several types of cancers including head-and-neck, ovarian and liver cancers. It is notable that IKKβ's role in cancer is not only limited to its function in the regulation of NF-κB pathway. IKKβ can also phosphorylate p53, which in turn results in its ubiquitination and subsequent degradation. Inactivation or loss of p53 has been identified in more than 50% of cancers, including HPV-induced cancers: HPV 18 and 16 E6 both target p53 for ubiquitination and proteasomal degradation. IKKβ-mediated loss of p53 can be suppressed by inhibition of IKKβ in cancer cells. Thus IKKβ is likely to contribute to tumorigenesis and cancer progression in several ways, some through the innate immune pathway, and some independent of it.

### Example 2: Auranofin Inhibits Growth of HPV 16⁺-Xenograft Tumors

Auranofin is a gold complex which has been used to treat rheumatoid arthritis since the 1980s. Auranofin has a well-established safety profile in human, and recently been investigated for its in vitro and in vivo anticancer activity in leukemia and various other cancers. The present example demonstrates the potent anti-cancerous activity of auranofin in cervical cancer in the absence of toxicity. These results support the development of auranofin for treatment of HPV-induced cervical cancer.

### Material and Methods

### Reagents

Auranofin was purchased from Sigma-Aldrich (#A6733-50MG, Saint Louis, Mo., USA) and dissolved in 2% DMSO, 10% ethanol and 5% polyethylene glycol 400 for the animal treatments.

### Tumor Xenograft Model

The animal study was conducted in accordance with a standard animal protocol approved by the University of Oklahoma Health Sciences Center Institutional Animal Care and Use Committee (IACUC Protocol #19-009-CHI). In brief, after an acclimation period, 4-5 week old female athymic Hsd: Athymic Nude-Foxn1^{nu} mice (ENVIGO, Alice, Tex., USA) were subcutaneously injected with 1.2×10⁷ SiHa cells (#HTB-35™, American Type Culture Collection (ATCC, Manassas, Va., USA) suspended in normal saline. Once tumors were palpable their volume was measured twice per week using calipers and the formula: 0.5 (width²×length). In this formula, the length is the larger diameter and the width is the smaller diameter. Once the tumor size achieved ˜50 mm³ average tumor volume, mice were randomized into two groups of 7 animals per treatment group based on tumor volume so that there were no significant differences between the groups (ANOVA, p>0.05). Auranofin (10 mg/kg/day) or placebo (2% DMSO, 10% ethanol and 5% polyethylene glycol 400) were given daily for 16 days, through intraperitoneal injection. Mice were weighed once per week and animal health was monitored daily. At the end of the study, all mice were euthanized by CO₂ inhalation followed by cardiac puncture and total blood collection. Tumors were collected at necropsy and a portion of each tumor from each animal was fixed in paraformaldehyde and embedded in paraffin, while another portion was snap-frozen in liquid nitrogen.

### Statistical Analysis

The tumor volume and body weight data were determined to be not normally distributed, and the final tumor weight data was determined to be normally distributed using the Shapiro-Wilk test. Tumor volume and body weight were compared between treatment groups using the Wilcoxon matched-pairs signed rank test. A two-way ANOVA with multiple comparisons was used to evaluate the interaction between time and treatment. An unpaired t-test was used to compare the final tumor weights between the two treatment groups. The p values <0.05 were considered statistically significant. Statistical analysis was performed using Prism 8.0 (GraphPad).

### Results

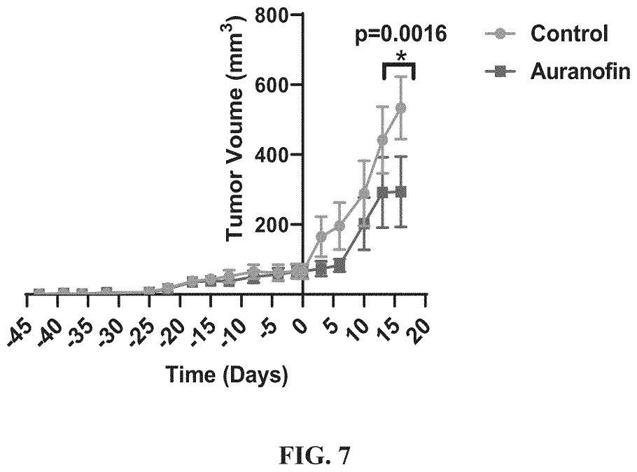

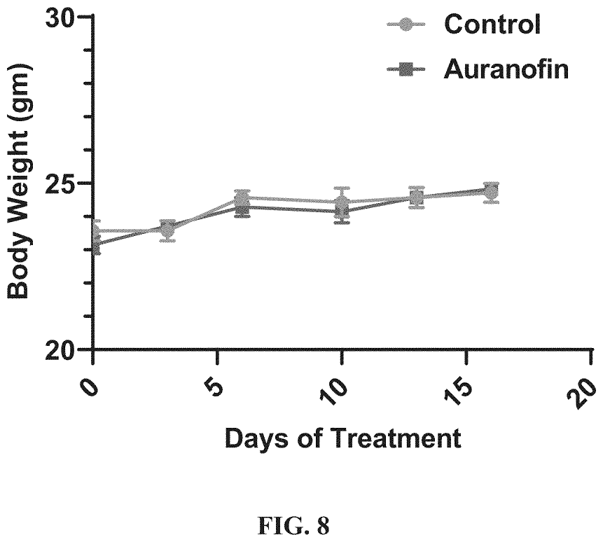

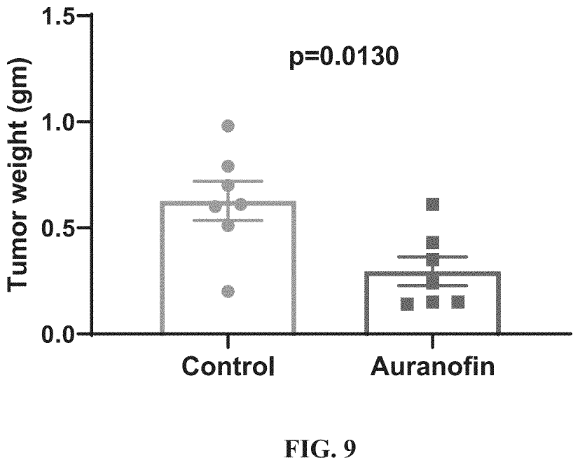

To investigate the anti-cancerous effect of auranofin in vivo, female mice harboring SiHa xenograft tumors were treated with placebo or 10 mg/kg auranofin by i.p. injection every day for 16 days. Auranofin induced a significant reduction in tumor growth (FIG. 7; Wilcoxon matched-pairs signed rank test, p=0.0011). There was a significant interaction between time and treatment (Two-way ANOVA, p=0.0217). After 16 days of treatment auranofin treatment group animals exhibited significant reduction in tumor volume (FIG. 8; Two-way ANOVA multiple comparisons: p=0.0016). The final tumor weight also was significantly different between the two treatment groups (FIG. 9; unpaired t test: p=0.0130). The average body weights and growth of the mice were consistent in both groups during the treatment period and there were no significant differences in body weights between the two groups at any time point (p>0.05) indicating that drug treatments did not cause gross toxicity to the animals.

While the present disclosure has been described herein in connection with certain embodiments so that aspects thereof may be more fully understood and appreciated, it is not intended that the present disclosure be limited to these particular embodiments. On the contrary, it is intended that all alternatives, modifications and equivalents are included within the scope of the present disclosure as defined herein. Thus the examples described above, which include particular embodiments, will serve to illustrate the practice of the inventive concepts of the present disclosure, it being understood that the particulars shown are by way of example and for purposes of illustrative discussion of particular embodiments only and are presented in the cause of providing what is believed to be the most useful and readily understood description of procedures as well as of the principles and conceptual aspects of the present disclosure. Changes may be made in the formulations, compounds, and compositions described herein, the methods described herein or in the steps or the sequence of steps of the methods described herein without departing from the spirit and scope of the present disclosure. Further, while various embodiments of the present disclosure have been described in claims herein below, it is not intended that the present disclosure be limited to these particular claims.

What is claimed is:

1. A method of treating a human papillomavirus (HPV)-induced cancer in a subject in need of such treatment comprising administering to the subject an inhibitor of Inhibitor of Nuclear Factor kappa-B kinase subunit beta (IKKβ).

2. The method of claim 1, wherein the HPV-induced cancer is selected from the group consisting of cervical, head and neck, mouth, tongue, oropharyngeal, anal, vulvar, vaginal, and penile cancers.

3. The method of claim 1, wherein the IKKβ inhibitor is selected from the group consisting of ACHP (IKK inhibitor VIII), Ainsliadimer A, anti-IKKβ antibody clone 10AG2, auranofin, Bay 65-1942, BI605906 (BIX02514), BMS-345541, BOT-64, CDDO-Me, IKK16 (IKK inhibitor VII), IMD-0354, IMD-1041, LY2409881, MLN120B, PF-184, PHA-408, PS-1145, SC-514, TPCA-1, Wedelactone, and Withaferin A.

4. A method of inhibiting growth of human papillomavirus (HPV)-induced cancer cells, comprising exposing the cells to an inhibitor of Inhibitor of Nuclear Factor kappa-B kinase subunit beta (IKKβ).

5. The method of claim 4, wherein the HPV-induced cancer is selected from the group consisting of cervical, head and neck, mouth, tongue, oropharyngeal, anal, vulvar, vaginal, and penile cancers.

6. The method of claim 4, wherein the IKKβ inhibitor is selected from the group consisting of ACHP (IKK inhibitor VIII), Ainsliadimer A, anti-IKKβ antibody clone 10AG2, auranofin, Bay 65-1942, BI605906 (BIX02514), BMS-345541, BOT-64, CDDO-Me, IKK16 (IKK inhibitor VII), IMD-0354, IMD-1041, LY2409881, MLN120B, PF-184, PHA-408, PS-1145, SC-514, TPCA-1, Wedelactone, and Withaferin A.

7. The method of claim 4, wherein the HPV-induced cancer cells are treated in vivo.

8. The method of claim 4, wherein the HPV-induced cancer cells are treated in vitro.
